(19) United States
(12) Patent Application Publication
(10) Pub. No.: US 2023/0305004 A1
(43) Pub. Date: Sep. 28, 2023

(54) POINT-OF-CARE DIAGNOSTIC FOR DETECTING THE NUCLEOCAPSID PROTEIN OF SARS-COV-2

(71) Applicant: The Regents of the University of California, Oakland, CA (US)

(72) Inventors: Daniel Takashi Kamei, Monterey Park, CA (US); Daniel William Bradbury, Los Angeles, CA (US); Ren Sun, Pacific Palisades, CA (US); Yushen Du, Los Angeles, CA (US); Benjamin Ming Wu, San Marino, CA (US); Jasmine Thanh Trinh, Alameda, CA (US); Milo Ryan, Larkspur, CA (US); Cassandra Marie Cantu, Sylmar, CA (US)

(73) Assignee: The Regents of the University of California, Oakland, CA (US)

(21) Appl. No.: 18/020,604

(22) PCT Filed: Aug. 16, 2021
(86) PCT No.: PCT/US2021/046166

§ 371 Date: Feb. 9, 2023

Related U.S. Application Data
(60) Provisional application No. 62/706,490, filed on Aug. 19, 2020.

(51) Int. Cl. G01N 33/569; G01N 33/543; C12M 1/34

(52) U.S. Cl. CPC G01N 33/56983; G01N 33/54388; C12M 1/34; G01N 33/54346; G01N 2333/165

(57) ABSTRACT

In various embodiments methods to improve the detection of a lateral-flow immunoassay for the sensitive detection of the SARS-CoV-2 nucleocapsid protein or other analytes, as well as devices that incorporate those methods are provided.

### CROSS-REFERENCE TO RELATED APPLICATIONS

This application claims benefit of and priority to U.S. Ser. No. 62/706,490, filed on Aug. 19, 2020, which is incorporated herein by reference in its entirety for all purposes.

### STATEMENT OF GOVERNMENTAL SUPPORT

This invention was made with government support under Grant Number 1707194, awarded by the National Science Foundation. The government has certain rights in the invention.

### BACKGROUND

The severe acute respiratory syndrome coronavirus 2 (SARS-CoV-2) has caused an ongoing and devastating pandemic which remains a major threat to global public health (Pokhrel et al. (2020) ACS Sensors, 5: 2283-2296; Wu et al. (2020) Int. J. Infect. Dis. 94: 44-48). The nucleocapsid protein (N-protein) is a major structural protein of coronaviruses which is involved in the packing of RNA within the virus. During the first week of infection, the N-protein is shed at relatively high concentrations into nasopharyngeal fluid and serum (Kammila et al. (2008) J. Virol. Met. 152: 77-84). It has previously been utilized to diagnose SARS-CoV infections, where the viral N-protein could be detected as early as 1 day after onset of symptoms in a variety of different bodily fluids (Che et al. (2004) Emerging Infect. Dis. 10: 1947-1949). Recent studies have shown that patients in the early stages of infection with SARS-CoV-2 also have detectable circulating N-protein in serum (Wang et al. (2020) Frontiers in Cellular and Infection Microbiology, 10: 470; Shan et al. (2021) Nat. Comm. 12: 1931). Therefore, it has been suggested that the N-protein in serum could serve as an antigen target for early SARS-CoV-2 detection.

An at-home diagnostic would allow for more widespread rapid detection of initial infection in a low-cost manner, which would allow patients to be treated and quarantined to prevent further outbreaks. Lateral-flow immunoassays (LFAs) exhibit many of the characteristics desired for point-of-care diagnostics and can easily be performed at home with the correct sampling method. The most common application of LFAs is the over-the-counter pregnancy test. Having a similar rapid, inexpensive, and easy-to-use test for SARS-CoV-2 will lead to widespread screening of healthy, asymptomatic, and symptomatic individuals. This blanket screening approach will play a significant role in allowing society to return to normal while maintaining safety.

Several LFAs that directly detect the viral antigens (both spike protein and N-protein) during the early stages of infection (within the first week of infection) have become commercially available during the pandemic through the FDA's Emergency Use Authorization (Linares et al. (2020) J. Clin. Virol. 133: 3-6; Santiago (2020) ChemBioChem, 21: 2880-2889; U.S. FDA In Vitro Diagnostics EUAs—Antigen Diagnostic Tests for SARS-CoV-2). It is important to note that all of the antigen-based LFAs currently available use nasal and/or nasopharyngeal swabs for sample collection. While the use of a nasopharyngeal swab has the potential to sample and capture the most virus due to the localization of SARS-COV-2 in the upper respiratory tract, it requires some level of guidance to ensure proper sample collection and thus is not ideal for at-home testing. Both swabbing collection methods are also prone to user error and variation depending on how the user inserts the swab into the nasal cavity. In fact, swabbing variability has been shown to impact even highly sensitive laboratory diagnostics for SARS-CoV-2 (Lippi et al. (2020) Clin. Chem. Lab. Med. 58: 1070-1076; Basso et al. (2020) Clin. Chem. Lab. Med. 58: 1579-1586). Additionally, nasal and nasopharyngeal swabs must be significantly diluted into a buffer before being utilized in any LFA-based diagnostic. These disadvantages cause the nasal and nasopharyngeal swabs to be less-than-ideal sample collection methods for an at-home diagnostic.

In contrast, sample fluids such as blood, serum, and saliva can be utilized and are easier to collect consistently. However, they cannot be used with the currently available LFA technology due to having lower viral loads or antigen concentrations than those in nasopharyngeal samples. Li and Lillehoj (2021) ACS Sensors, 6: 1270-1278, recently reported the development of the first smartphone-based, microfluidic point-of-care device for the sensitive quantification of N-protein in serum down to 0.1 ng/mL. While able to detect low levels of N-protein, this assay requires the user to perform multiple reagent addition steps and possess a smartphone, potentially limiting its applicability for self-testing and widespread use in low resource regions (Li & Lillehoj (2021) ACS Sensors, 6: 1270-1278). An alternative approach is to develop a more sensitive version of the LFA which maintains its ease-of-use and equipment-free characteristics while also being able to detect low levels of N-protein in blood. Some common techniques to improve LFA sensitivity involve biomarker preconcentration and signal enhancement (Liu et al. (2021) Biosensors and Bioelectronics, 173: 112817; Bradbury et al. (2019) Anal. Chem. 91: 12046-12054).

### SUMMARY

As described herein, a novel paper-based device was developed that incorporates an LFA test strip, dehydrated signal enhancement reagents (e.g., nanozymes and their associated chemicals), and a sealed chamber with stored liquid enhancement buffer in an innovative casing that can easily be 3-D printed. The device enabled the detection of N-protein in undiluted serum in 40 min at concentrations as low as 0.1 ng/mL, which was at least a 10-fold improvement over the conventional LFA. Moreover, with this all-in-one device, only one simple step of pushing a single button is needed for the signal enhancement to occur after the LFA detection step. Additionally, while the device and methods are described herein with respect to detection of N-protein, it will be recognized that they can readily be applied to detection of a number of other analytes.

Accordingly, various embodiments provided herein may include, but need not be limited to, one or more of the following:

Embodiment 1: A device for performing a signal-enhanced lateral flow immunoassay to detect an analyte, said device comprising:”
a case comprising a top piece, a middle piece, and a bottom piece where said casing is configured so provide that the middle piece can move from a first position to a second position, wherein:
   said bottom piece comprises a lateral flow immunoassay strip configured to detect said analyte, one or more enhancement reagent pads comprising enhancement reagent(s) disposed on or within said enhancement reagent pads, an enhancement reagent absorbent pad configured to receive said enhancement reagent(s), and a well or receptacle configured to deliver a buffer into said one or more pads comprising enhancement reagent(s);
   said top piece comprises an opening to a sample well for receiving a sample and a viewing window that permits viewing of a detection zone on said lateral flow immunoassay strip; and
   said middle piece comprises a reservoir that contains an enhancement reagent buffer, one or more connector channels and/or connector pads configured to carry said enhancement reagent buffer and said one or more enhancement reagents to and across said lateral flow immunoassay strip to said enhancement reagent absorbent pad, and one or more tabs or a button to move said middle piece from said first position to said second position; and wherein:
   in said first position, said channels and/or connector pads are not in fluid communication with said lateral flow immunoassay strip and said reservoir is sealed; and
   in said second position, said reservoir is in fluid communication with well which is in fluid communication with said one or more channels and/or connector pads, said one or more pads comprising enhancement reagents, and said lateral flow immunoassay strip and thereby capable of delivering said enhancement reagent buffer and said one or more enhancement reagents to said lateral flow immunoassay strip.

Embodiment 2: The device of embodiment 1, wherein said analyte comprises a nucleocapsid protein (N-protein) of a corona virus.

Embodiment 3: The device of embodiment 2, wherein said analyte comprises a nucleocapsid protein (N-protein) of SARS-CoV-2 (Covid 19).

Embodiment 4: The device according to any one of embodiments 1-3, wherein said lateral flow assay strip comprise a sample pad, a detection zone downstream from the sample pad, and an absorbent pad downstream from the detection zone, and where said detection zone comprises a test line comprising biotin or avidin.

Embodiment 5: The device of embodiment 4, wherein said lateral flow assay strip comprises a capture moiety disposed in said LFA strip upstream from said detection zone and said capture moiety comprises a first anti-analyte antibody conjugated to a biotin when said test line comprises an avidin and said capture moiety comprises a first anti-analyte antibody conjugated to an avidin when said test line comprises a biotin.

Embodiment 6: The device of embodiment 5, wherein said anti-analyte antibody is conjugated to a biotin and said test line comprises an avidin.

Embodiment 7: The device of embodiment 6, wherein said test line comprises streptavidin, or polystreptavidin.

Embodiment 8: The device according to any one of embodiments 4-7, wherein said detection zone comprises a control line functionalized to capture an antibody.

Embodiment 9: The device of embodiment 8, wherein said control line comprises an anti-IgG antibody.

Embodiment 10: The device according to any one of embodiments 5-9, wherein said capture moiety is dried onto or into said lateral flow assay strip or onto or into a pad disposed on said lateral flow assay strip.

Embodiment 11: The device according to any one of embodiments 5-10, wherein said capture moiety is disposed on said lateral flow assay strip between said sample pad and said detection zone or on a pad (e.g., nanozyme conjugate pad) disposed on said lateral flow assay strip between said sample pad and said detection region.

Embodiment 12: The device according to any one of embodiments 5-10, wherein said capture moiety is disposed on or in said sample pad.

Embodiment 13: The device according to any one of embodiments 5-12, wherein said first anti-analyte antibody comprises an anti-N-protein antibody.

Embodiment 14: The device according to any one of embodiments 5-12, wherein said lateral flow assay comprises a region containing an indicator conjugate comprising an indicator moiety attached to a second anti-analyte antibody or wherein said lateral flow assay comprises a conjugate pad (e.g., nanozyme conjugate pad) containing said indicator conjugate.

Embodiment 15: The device of embodiment 14, wherein said second anti-analyte antibody comprises an anti-N-protein antibody.

Embodiment 16: The device according to any one of embodiments 14-15, wherein said indicator moiety comprises an indicator selected from the group consisting of a nanozyme, horseradish peroxidase, alkaline phosphatase, and a gold nanoparticle.

Embodiment 17: The device according to any one of embodiments 14-16, wherein said indicator conjugate is disposed on said lateral flow assay strip between said sample pad and said detection region or on a pad (e.g., nanozyme conjugate pad) disposed on said lateral flow assay strip between said sample pad and said detection region.

Embodiment 18: The device according to any one of embodiments 14-17, wherein said indicator conjugate is disposed on or in said lateral flow assay strip downstream from said capture moiety or on or in a pad disposed on said lateral flow downstream from said capture moiety.

Embodiment 19: The device according to any one of embodiments 1-18, wherein said bottom piece comprises one pad with one or more enhancement reagent(s) disposed on or within said pad.

Embodiment 20: The device according to any one of embodiments 1-18, wherein said bottom piece comprises two pads with one or more enhancement reagent(s) disposed on or within each pad.

Embodiment 21: The device according to any one of embodiments 19-20, wherein the enhancement reagent(s) are dried onto said enhancement reagent pad(s).

Embodiment 22: The device according to any one of embodiments 16-21, wherein said indicator conjugate comprises a nanozyme conjugate comprising a nanozyme attached to said second anti-analyte antibody.

Embodiment 23: The device of embodiment 22, wherein said nanozyme comprises a material selected from the group consisting of platinum, gold, iron oxide, cerium-oxide, rubidium, iridium, copper, and palladium.

Embodiment 24: The device of embodiment 23, wherein said nanozyme comprises a nanoparticle selected from the group consisting of a platinum-coated gold nanoparticle, and Fe₃O₄ nanoparticle, a palladium core-shell nanoparticle, a Pt core shell nanoparticle, and a Pd/Pt core shell nanoparticle.

Embodiment 25: The device of embodiment 24, wherein said nanozyme comprises a platinum-coated gold nanoparticle.

Embodiment 26: The device according to any one of embodiments 22-25, wherein said one or more enhancement reagent pads contains a peroxidase substrate selected from the group consisting of tetramethylbenzidine (TMB), diaminobenzidine (DAB), ABTS peroxidase substrate (Cas No: 28752-68-3), and o-phenylenediamine dihydrochloride (OPD), chemiluminescent luminol, a xanthan ester, and an acridan-based reagent.

Embodiment 27: The device of embodiment 26, wherein said peroxidase substrate comprises TMB.

Embodiment 28: The device according to any one of embodiments 16-21, wherein said indicator moiety comprises alkaline phosphatase.

Embodiment 29: The device of embodiment 28, wherein said one or more enhancement reagent pads contains a substrate selected from the group consisting of nitroblue tetrazolium/5-bromo-4-chloro-3-indolyl phosphate (NBT/BCIP), and p-Nitrophenyl Phosphate, Disodium Salt (PNPP).

Embodiment 30: The device according to any one of embodiments 16-21, wherein said indicator moiety comprises a gold nanoparticle.

Embodiment 31: The device of embodiment 30, wherein said one or more enhancement reagent pads contains a substrate selected from the group consisting of gold (III) chloride, and silver nitrate.

Embodiment 32: The device according to any one of embodiments 1-31, wherein said lateral flow assay strip, said LFA absorbent pad, said LFA sample pad, said one or more reagent pads, and said enhancement reagent absorbent pad each comprise a paper.

Embodiment 33: The device of embodiment 32, wherein said the paper comprising wherein said lateral flow assay strip, said LFA absorbent pad, said LFA sample pad, said one or more reagent pads, and said enhancement reagent absorbent pad are independently selected from the group consisting of a cellulose paper, a fiberglass paper, a nitrocellulose membrane, a polyvinylidene fluoride, a nylon membrane, a charge modified nylon membrane, a polyethersulfone, and a cotton linter material (e.g., CF4), and combinations thereof.

Embodiment 34: The device according to any one of embodiments 32-33, wherein said lateral flow assay strip comprises a nitrocellulose membrane.

Embodiment 35: The device according to any one of embodiments 32-34, wherein lateral flow assay absorbent pad comprises a cotton linter material (e.g., CF4).

Embodiment 36: The device according to any one of embodiments 32-35, wherein said sample pad comprises a fiberglass paper.

Embodiment 37: The device according to any one of embodiments 32-36, wherein said indicator conjugate is disposed in a pad comprising a fiber glass paper.

Embodiment 38: The device according to any one of embodiments 32-37, wherein said enhancement reagent pad(s) each comprise a fiberglass paper.

Embodiment 39: The device according to any one of embodiments 1-38, wherein:
said middle piece comprises a first connector pad configured so that, when said middle piece is in the second position said first connector pad is upstream from said lateral flow assay strip with respect to the flow of said enhancement reagent buffer and provides fluid communication from said enhancement reagent pad(s) to said lateral flow assay strip; and
said middle piece comprises a second connector pad configured so that, when said middle piece is in the second position said second connector pad is downstream from said lateral flow assay strip with respect to the flow of said enhancement reagent buffer and provides fluid communication from said lateral flow assay strip to said enhancement reagent absorbent pad.

Embodiment 40: The device of embodiment 39, wherein said first connector pad and said second connector pad each comprise a paper.

Embodiment 41: The device of embodiment 40, wherein said the paper comprising said first connector pad and said second connector pad are independently selected from the group consisting of a cellulose paper, a fiberglass paper, a nitrocellulose membrane, a polyvinylidene fluoride, a nylon membrane, a charge modified nylon membrane, a polyethersulfone, and a cotton linter material (e.g., CF4), and combinations thereof.

Embodiment 42: The device according to any one of embodiments 40-41, wherein said first connector pad comprises a fiberglass paper.

Embodiment 43: The device according to any one of embodiments 40-42, wherein said second connector pad comprises a cotton linter material.

Embodiment 44: The device according to any one of embodiments 1-38, wherein:
said middle piece comprises a first connector channel configured so that, when said middle piece is in the second position said first connector channel is upstream from said lateral flow assay strip with respect to the flow of said enhancement reagent buffer and provides fluid communication from said enhancement reagent pad(s) to said lateral flow assay strip; and
said middle piece comprises a second connector channel configured so that, when said middle piece is in the second position said second connector channel is downstream from said lateral flow assay strip with respect to the flow of said enhancement reagent buffer and provides fluid communication from said lateral flow assay strip to said enhancement reagent absorbent pad.

Embodiment 45: The device according to any one of embodiments 1-44, wherein said well or receptacle configured to deliver a buffer into said one or more pads comprising enhancement reagent(s) comprises is an enclosed hollow cylinder with a dome or cone in the center configured to puncture a seal on said reservoir when said middle piece is in the second position and thereby release enhancement reagent buffer into said well or receptacle.

Embodiment 46: The device according to any one of embodiments 1-45, wherein said reservoir is sealed with a foil seal.

Embodiment 47: The device according to any one of embodiments 1-46, wherein said top piece and said bottom piece are snapped together.

Embodiment 48: The device according to any one of embodiments 1-47, wherein said middle piece comprises a push button that protrudes through said top piece and operates to move said middle piece from said first position to said second position.

Embodiment 49: The device according to any one of embodiments 1-47, wherein said middle piece comprises tabs that protrude through sides of said device said and operates to move said middle piece from said first position to said second position.

Embodiment 50: A method of detecting an analyte, said method comprising:
applying a sample comprising said analyte to a lateral flow assay configured for the capture of said analyte at a test line and permitting said analyte to flow through said lateral flow assay; and
contacting said lateral flow with signal enhancement reagents to enhance and improve visibility of a signal at said test line.

Embodiment 51: The method of embodiment 50, wherein said enhancement reagents comprise a nanozyme.

Embodiment 52: The method according to any one of embodiments 50-51, wherein said method is performed using a device according to any one of embodiments 1, and 4-49, wherein said method comprises:
applying said sample to a sample well on said device when said second piece is in said first position;
allowing said sample to pass through said lateral flow assay; and
moving said second piece from said first position to said second position and allowing said enhancement reagents to react on bound enhancement reagents (e.g., nanozyme) on said test line; and detecting a visible line at said test line that indicates the presence of said analyte.

Embodiment 53: The method of embodiment 52, wherein said analyte comprises a nucleocapsid protein (N-protein) of a corona virus.

Embodiment 54: The method of embodiment 53, wherein said analyte comprises a nucleocapsid protein (N-protein) of SARS-CoV-2 (Covid 19).

Embodiment 55: The method according to any one of embodiments 50-54, wherein said sample comprises a biological sample.

Embodiment 56: The method of embodiment 55, wherein said sample comprises a biological sample selected from the group consisting of a culture, blood, serum, saliva, nasal mucus, cerebral spinal fluid, urine, stool, bronchial aspirates, tracheal lavage, pleural fluid, milk, lymph, sputum, semen, needle aspirate, punch biopsy, and surgical biopsy.

Embodiment 57: The method of embodiment 56, wherein said sample comprises a biological sample selected from the group consisting of nasal mucus, oral fluid, bronchial aspirate, and trachial lavage.

### BRIEF DESCRIPTION OF THE DRAWINGS

FIG. 1 shows a schematic illustration of a typical lateral-flow immunoassay (LFA) test strip (top), and an illustration of the sandwich format of the lateral-flow immunoassay. In the embodiment illustrated, the test strip 100 comprises a paper strip 104 (e.g., a nitrocellulose membrane), a sample pad 102 for receiving a sample, an absorbent pad 106, and a detection zone/region 113, comprising a detection line (signal line) 110, and a control line 112.

FIG. 2 illustrates detection of the nucleocapsid protein of SARS-CoV-2 in serum. The detection limit before enhancement was 10 ng/mL while after enhancement it was 1 ng/mL, demonstrating a 10-fold improvement in detection limit.

FIG. 3 illustrates a design of one embodiment of a lateral flow assay device 320. In the embodiment illustrated the device comprises three main pieces (a top piece 322, a middle piece 324, and a bottom piece (e.g., base) 326) that are configured to provide for signal enhancement of the LFA while reducing user interactions and improving ease of use.

FIG. 4 shows a CAD drawing showing the underside view of the middle piece 424 of the casing. The locations of the enhancement buffer fluid reservoir 428, and the paper connector pads (430a, and 430b) are visible in this perspective. Also illustrated are tabs (432a, and 432b) that facilitate movement of the middle piece during use.

FIG. 5 shows a CAD drawing of the bottom piece 526 of the casing. The illustrated embodiment shows locations for a buffer release region 536, enhancement reagent pads (538a, and 538b), and an enhancement reagent absorbent pad 534. Also shown in the LFA comprising a first sample pad 502, a detection region comprising detection (signal) line 510 and control line 512, and an LFA absorbent pad 506. Also illustrated are alignment/joining tabs (540a, and 504b) that provide alignment of the various pieces of the device and allow the device to be snapped together.

FIG. 6 shows a CAD drawing of fully assembled casing 620 with the middle piece in its starting position, prior to pressing it down to initiate signal enhancement. Also illustrated are the tabs (632a, and 632b) that allow the middle piece to be moved into a downward position, a sample well (sample receiving well) 642, and a viewing window 644.

FIG. 7 shows a simplified schematic representation of the signal enhanced assay steps with locations of the paper segments touching the LFA test strip 700 shown. The enhancement reagent connector pad(s) 730a, and 730b are shown. When the middle piece is moved into the downward position, enhancement reagent(s) move along the enhancement reagent flow path 746 and contact the signal line 710 and the control line 712.

FIG. 8 illustrates CAD drawings showing the casing before and after pressing the middle piece down to initial the signal enhancement reaction.

FIG. 9 illustrates 3D-printed middle and bottom casing pieces to store liquid reagents in a sealed well (e.g., a mylar sealed well). Once the top piece is pressed down, the fluid is released, and the flow path is connected across the LFA test strip to deliver reagents to enhance signal. The locations of the enhancement buffer reservoir 936, the connector pads (930a, and 930b), the enhancement regent pad(s) (938a, and 938b), the LFA test strip 948, and the enhancement reagent absorbent pad 934 are shown.

FIG. 10 shows a design of another illustrative embodiment of a lateral flow assay device 1020. In the embodiment illustrated the device comprises three main pieces (a top piece 1022, a middle piece 1024, and a bottom piece (e.g., base) 1026) that are configured to provide for signal enhancement (e.g., nanozyme signal enhancement) of the LFA while reducing user interactions and improving ease of use.

FIG. 11, panel A shows a labelled CAD drawing of bottom piece of casing shown in FIG. 10. The figure shows locations for a well 1136 for the enhancement reagent buffer release, enhancement reagent pad(s) (1138a, and 1138b), an enhancement reagent absorbent pad 1134, and an LFA comprising a sample pad 1102, an LFA detection region 1113 comprising a signal line and optionally a control line, a biotinylated antibody pad 1102a, and a nanozyme conjugate pad 1102b. It will be recognized that, in certain embodiments, the sample receiving pad 1102 can be combined with the biotinylated antibody pad 1102a and, optionally the nanozyme conjugate pad 1102b. FIG. 11, panel B, shows a photograph of 3D printed bottom piece of casing with LFA test strip and enhancement reagent paper pads in position.

FIG. 12, panel A, shows a CAD drawing showing the underside view of the middle piece 1224 of the casing and the locations of the enhancement buffer fluid reservoir 1228, the foil 1229 sealing the reservoir, and the paper connector pads (1230a, and 1230b). FIG. 12, panel B, shows a photograph of the 3D printed middle piece 1224.

FIG. 13, panel A, shows a CAD drawing (top) and a photograph (bottom) of the 3D printed full casing assembly with viewing window 1344 and sample well 1342 that comprise the sample pad of the LFA. A US quarter is included for size comparison. Panel B provides CAD drawings showing the casing before and after pressing the button 1350 that moves the middle piece.

FIG. 14 provides a simplified schematic of assay steps and paper segments touching the LFA test strip. (1) Sample is applied to the sample well above the test strip where biotinylated antibody and PtGNPs are rehydrated and antigen capture occurs at the detection zone. (2) After pressing the button to move the middle piece of the casing down, enhancement buffer is released to rehydrate the dehydrated enhancement reagents and flow through the test strip resulting in signal enhancement at the detection zone.

FIG. 15 shows detection of the N-protein of SARS-CoV-2 in human serum using nanozyme signal enhanced LFA. The detection limit before enhancement was 1 ng/mL while after enhancement it was 0.1 ng/mL, demonstrating at least a 10-fold improvement in detection limit and detection of N-protein within the desired concentration range.

FIG. 16 shows a plot of relative test line signal intensity versus N-protein concentration for both the LFA (●) and enhancement steps (▴). Data is represented as the mean±SD (n=3).

### DEFINITIONS

The terms “lateral flow assay”, “LFA”, and lateral flow immunoassay refer to devices configured to detect the presence of a target substance in a liquid sample. LFAs operate by running a sample in the pad with reactive molecules that show a visual positive or negative result. The pads are based on a series of capillary beds, such as pieces of porous paper

The terms “test line”, and “signal line” are used interchangeably and refer to the region of a lateral flow assay that produces a signal when an analyte is present. Typically, although not necessarily, the signal will be a visible signal, e.g., a colorimetric signal.

The term analyte refers to any moiety that is to be detected. Analytes include, but are not limited to particular biomolecules (proteins, antibodies, nucleic acids), bacteria or components thereof, viruses or components thereof (e.g., coat proteins), fungi or components thereof, protozoa or components thereof, drugs, toxins, food pathogens, and the like.

The term “paper”, as used herein, is not limited to thin sheets from the pulp of wood or other fibrous plant substances although, in certain embodiments, the use of such papers in the devices described herein is contemplated. Papers more generally refer to porous materials often in sheet form, but not limited thereto that allow a fluid to flow through. Illustrative papers include, but are not limited to a cellulose paper, a fiberglass paper, a nitrocellulose membrane, a polyvinylidene fluoride, a nylon membrane, a charge modified nylon membrane, a polyethersulfone, a cotton linter material (e.g., CF4), and combinations thereof.

A “nanozyme” refers to a nanoparticle that has catalytic activity.

### DETAILED DESCRIPTION

Described herein is a rapid at-home, point-of-care (POC) test for the detection of the nucleocapsid protein (N-protein) of SARS-CoV-2 or other analytes. Although lateral-flow immunoassays (LFAs) have been and are being commercially developed for antibodies against SARS-CoV-2 (produced after the first week of infection), LFAs that directly detect the antigen during the early stages of infection (within the first week of infection) are not yet commercially available.

Such a diagnostic would allow for rapid detection of initial infection in a low-cost manner, which would allow patients to be treated and quarantined to prevent further outbreaks. LFAs can easily be performed at home, and its most common application is the over-the-counter pregnancy test. Such a rapid, inexpensive, and easy-to-use test will lead to widespread screening of healthy, asymptomatic, and symptomatic individuals. This blanket screening approach will play a significant role in allowing society to return to normal while maintaining safety.

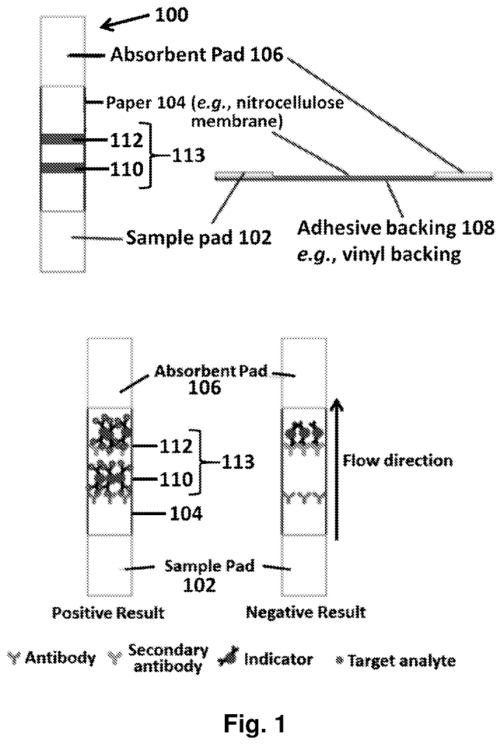

A typical LFA consists of at least 3 main components: a sample pad where the sample is applied to the test strip, a detection zone where there is binding and where results can be observed, and an absorbent pad (absorbent LFA pad) that acts as a sink for excess sample (FIG. 1). In the sandwich assay format, the sample is first mixed with an LFA indicator (which can be colorimetric, fluorescent, radioactive, etc.) decorated with binding molecules (often antibodies, aptamers, single-stranded DNA, etc.) and applied to the sample pad. In certain embodiments, however, the LFA indicator can be provided in a sample pad (e.g., dehydrated in a sample pad) or provided in a conjugate pad (e.g., dehydrated in a conjugate pad downstream from a sample pad). The sample pad often stores dehydrated buffers as well as blocking components to aid even flow of the sample throughout the strip. If the target analyte is present, it will bind to the indicator decorated with the antibodies. As these complexes continue to flow through the test strip, the analyte will also bind to the binding molecules immobilized on the test line and become sandwiched between the indicator and the membrane. If the indicator is colorimetric, the colorimetric indicator will exhibit a strong color, and a visual band forms as the analyte-indicator complex accumulates at the test line, indicating a positive result. Alternatively, if no analyte is present, the indicator does not attach to the test line, and the absence of the test line indicates a negative result. Regardless of the presence of the analyte, the binding molecule decorated on the indicator can bind and accumulate on the control line. A band at the control line signifies that the sample has flowed through the strip, indicating a valid test. A positive result is therefore typically indicated by two bands, one at the test line and one at the control line, while a negative result is indicated by a single band at the control line.

Described herein are methods to improve the detection of the lateral-flow immunoassay for the sensitive detection of the SARS-CoV-2 nucleocapsid protein as well as devices that incorporate those methods. Additionally, while the devices are described herein with respect to the detection of SARS-Cov2 antigen (e.g., N protein) it will be recognized that the devices can readily be used for the detection of other analytes (e.g., human chorionic gonadotropin (hCG), etc.).

In certain embodiments, the improved detection is accomplished through two approaches to integrate the LFA with signal enhancement reactions in order to amplify the intensity of the test line. One approach is a fully manual one that involves the movement of the test strip into various solutions in a multi-step manner. The second approach involves developing a casing design to eliminate some of the liquid handling steps. Both approaches are discussed herein, beginning with the multi-step approach. In this approach, as described in the example below, to detect a N-protein, an N-protein LFA is integrated with catalytic platinum-coated gold nanoparticles and common peroxidase substrates.

To synthesize platinum-coated gold nanozymes (PtGNs), the following protocol was used. First, 4 mL of 0.15 nM 40 nm citrate-capped gold nanoparticles (GNs) and 1827 μL of filtered ultrapure water were preheated to 90° C. in an oil bath with magnetic stirring for 20 min. Following the preheating, 173 μL of a 0.82 mM chloroplatinic acid hydrate solution and 2 mL of a 3.3 mM ascorbic acid solution were injected separately into the gold nanoparticle suspension using a syringe pump at rates of 0.6 and 1.2 mL/h, respectively. The reaction was allowed to proceed for 30 min after the injection was complete.

To create anti-N-protein platinum-coated gold nanozyme probes (PtGNPs), 7.5 μL of a 0.1 M sodium borate solution (pH 9) was first added to 0.5 mL of PtGNs. Then, 2 μg of anti-N-protein antibody was added to the suspension and incubated for 30 min at room temperature (22° C.). 50 μL of a 10% (w/v) bovine serum albumin (BSA) solution was then added to the suspension and incubated for 10 min. Free antibodies were removed by centrifugation and the pellet was resuspended in 50 μL of 0.1 M citrate buffer (pH 6) solution.

The capture antibody was biotinylated using the following procedure. First, a 20 mM solution of NHS-PEG-Biotin linker was made in dimethylformamide (DMF). This stock was diluted in Milli-Q water resulting in a 1 mM solution of NHS-PEG-Biotin. 9 μL of 1 mM NHS-PEG-Biotin was added to 100 μL of 0.5 mg/mL capture antibody and allowed to react for 30 min on an orbital shaker. Free, unconjugated linker was removed through purification with a 0.5 mL 7k MWCO zeba desalting column.

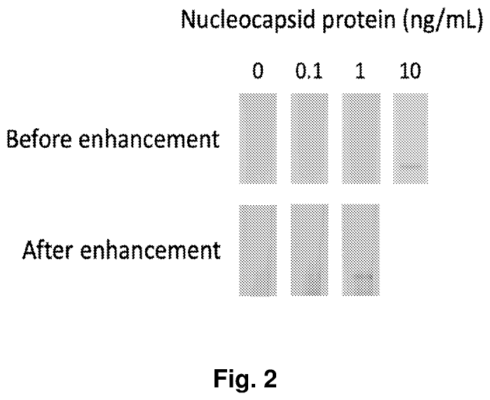

The LFA strips in this example were composed of overlapping pads secured to an adhesive backing. These pads included a sample pad, a nitrocellulose membrane, and a cotton fiber absorbent pad. In our work, the sample pad consisted of a 3 mm×10 mm piece of fiberglass paper. LFA strips utilized the double sandwich assay format where polystreptavidin was immobilized on a nitrocellulose membrane at 2 mg/mL. This constituted the test line of the test strip. The experiment that led to the results in FIG. 2 did not include a control line. Test line printing was performed using an Automated Lateral Flow Reagent Dispenser and a Fusion 200 syringe pump with a flow rate of 300 μL/min. The membrane was left in a vacuum-sealed desiccation chamber overnight. The nitrocellulose membrane was placed on the adhesive backing and cut into strips 3 mm in width prior to assembly with the sample pad and absorbent pad.

To detect the N-protein in serum using the multistep enhanced LFA, the test strip was inserted vertically into a solution composed of 25 μL of the serum sample, 20.5 μL of running buffer (0.2% BSA, 0.2% Tween 20, 2% polyvinylpyrrolidone, 0.2% casein in 100 mM phosphate buffer, pH 7.3), 2 μL of biotinylated anti-N-protein capture antibody, and 2.5 μL of anti-N-protein PtGNPs. In the case of a positive test, the N-protein would become sandwiched between the biotinylated capture antibody and the PtGNPs. Subsequently, as the complex flowed past the test line, it would become captured by the polystreptavidin immobilized on the membrane. After 10 min, the strip was transferred into an enhancement solution containing 0.16 M hydrogen peroxide, 1.3 mM 3,3′,5,5′-tetramethylbenzidine (TMB), and 0.1% (w/v) dextran sulfate in 0.1 M citrate buffer (pH 5). As the enhancement solution flowed past the test line, any PtGNPs bound to the test line region would catalyze the oxidation of the TMB substrate by the hydrogen peroxide. The interaction between the oxidized TMB product and dextran sulfate would result in the deposition of a dark purple precipitate at the test line which would enhance the visible signal. Results were interpreted after 30 min of enhancement. The results shown in FIG. 2 demonstrate the use of this multistep technique for the detection of N-protein in serum before and after the enhancement step. The N-protein was successfully detected down to 10 ng/mL before enhancement and 1 ng/mL (within the clinically relevant range) after enhancement, indicated by the formation of a visible test line at those concentrations. Thus, the signal enhancement resulted in a 10-fold improvement in the detection limit of the N-protein.

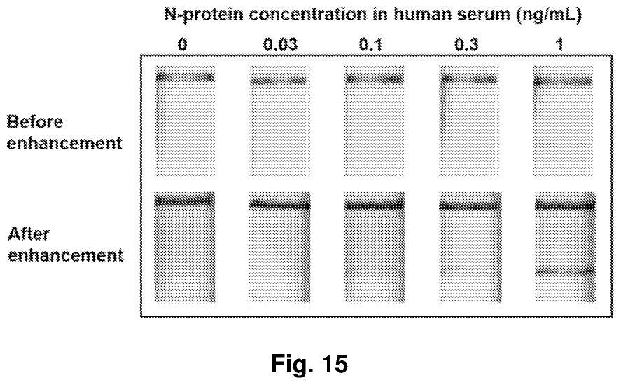

Another illustrative, but non-limiting embodiments is illustrated in FIG. 15 and described in detail in Example 1. In the illustrated embodiment, analyte detection a concentrations as low as 0.1 ng/mL have been achieved.

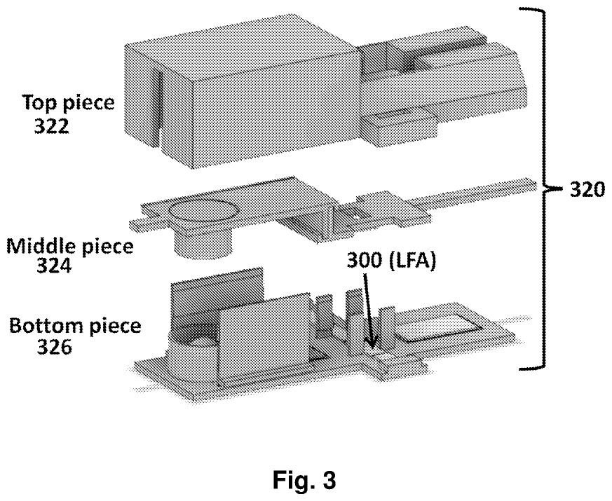

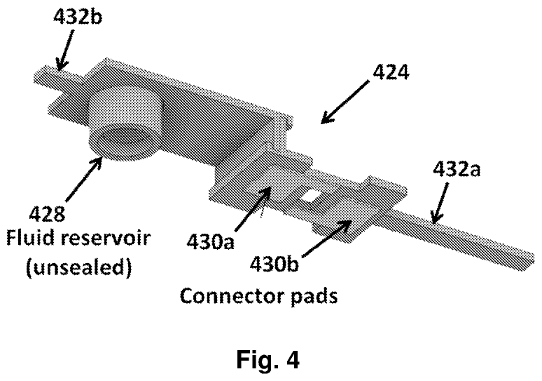

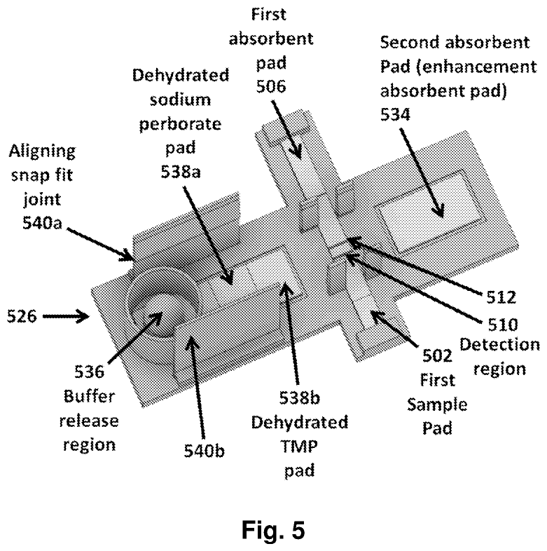

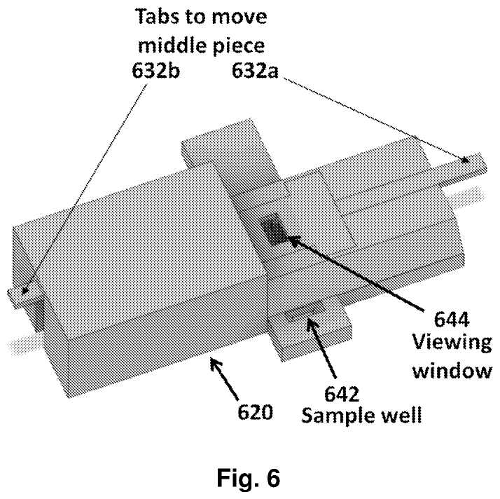

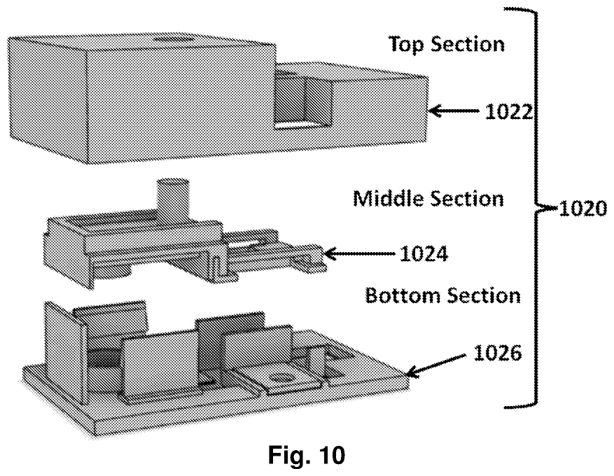

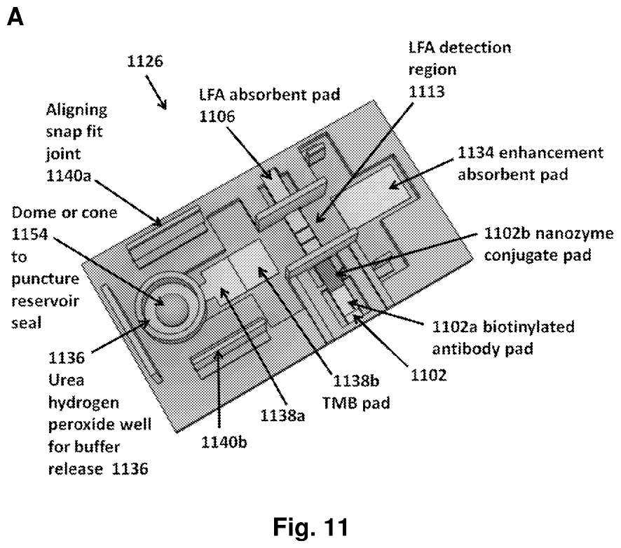

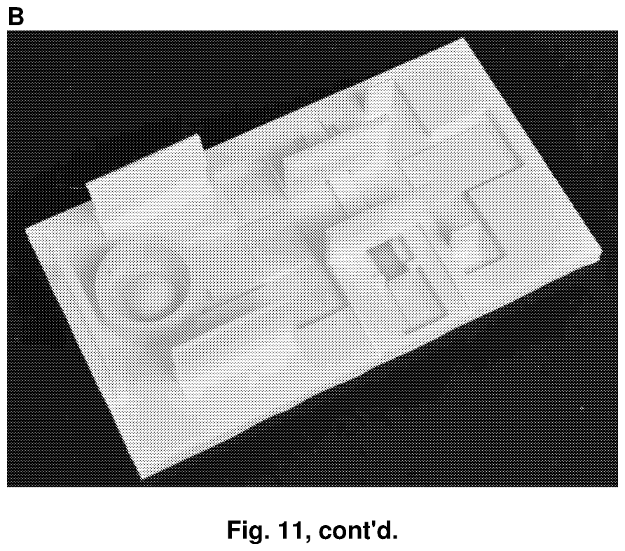

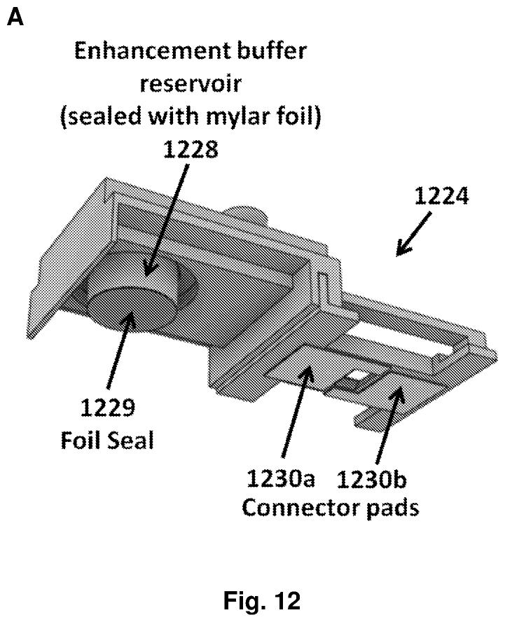

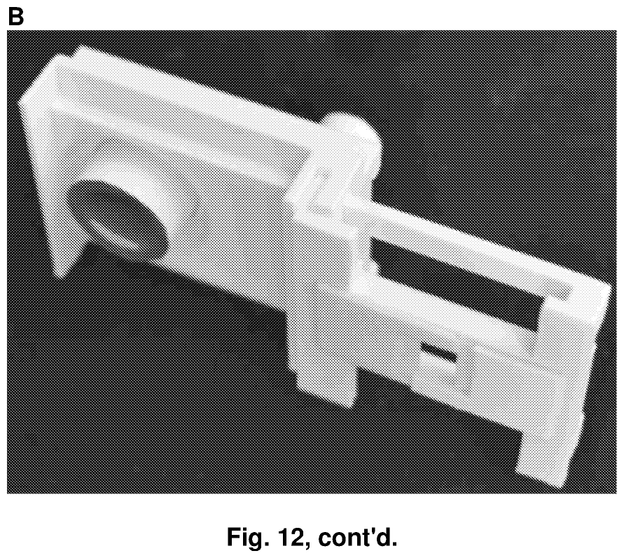

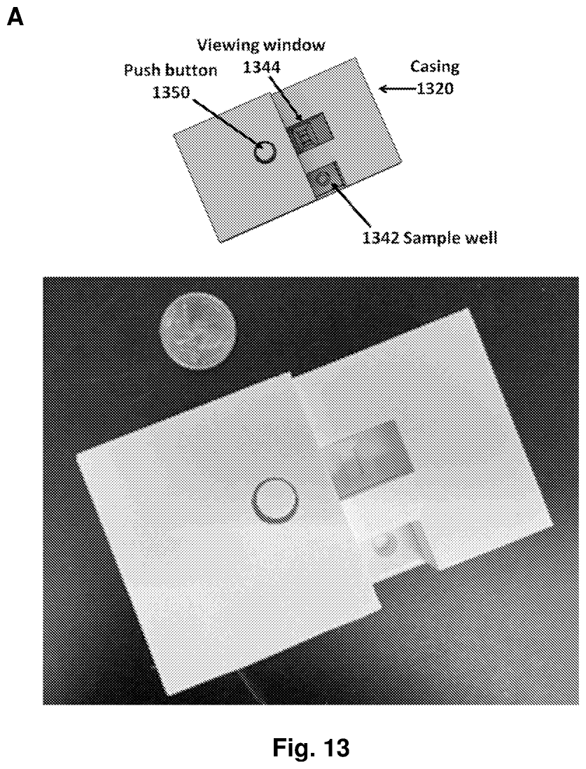

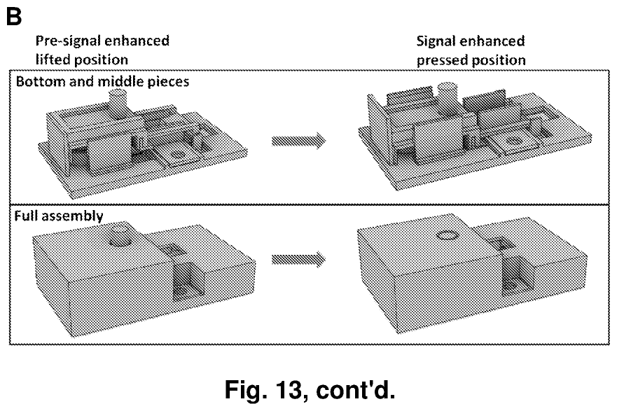

To eliminate the need for multiple liquid- and test strip-handling steps, a plastic casing was developed with onboard liquid reagent storage, dehydrated enhancement reagents, and movable paper architecture to direct the flow of liquid through the test strips. FIGS. 3 and 10 outlines illustrative embodiments of the casing (320 in FIG. 3, and 1020 in FIG. 10). The top piece of the casing (322 in FIG. 3, and 1022 in FIG. 10) serves to help hold the other components in place and protect them from external and environmental factors. The movable middle piece (324 in FIG. 3, and 1024 in FIG. 10), shown in more detail in FIG. 4 and FIG. 12, panels A and B, comprises a fluid reservoir (428 in FIG. 4, and 1228 in FIG. 12, panel A) that can be sealed, e.g., heat sealed with a mylar foil (1229 in FIG. 12, panel A), and connector pads (e.g., glass fiber connector pads) (430a and 430b FIG. 4, and 1230a and 1230b in FIG. 12, panel A). The bottom pieces, 526 detailed in FIG. 5, and 1126 detailed in FIG. 11, panel A, houses the LFA test strip, pads with dehydrated enhancement reagents, a second absorbent pad for the enhancement step, and a region to release the fluid sealed in the reservoir of the middle piece. The fully assembled casing in the pre-enhancement position is shown in FIG. 6 and FIG. 13, panel A, where the sample well (642 in FIG. 6 and 1342 in FIG. 13, panel A), viewing window (644 in FIG. 6 and 1344 in FIG. 13, panel A), and tabs (632a and 623b in FIG. 6) or a push button (1350 in FIG. 13, panel A) to move the middle piece are defined.

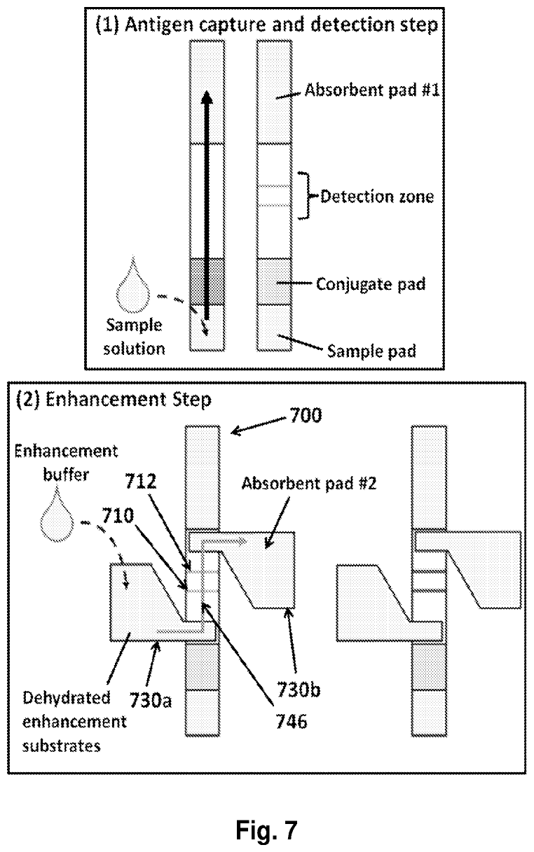

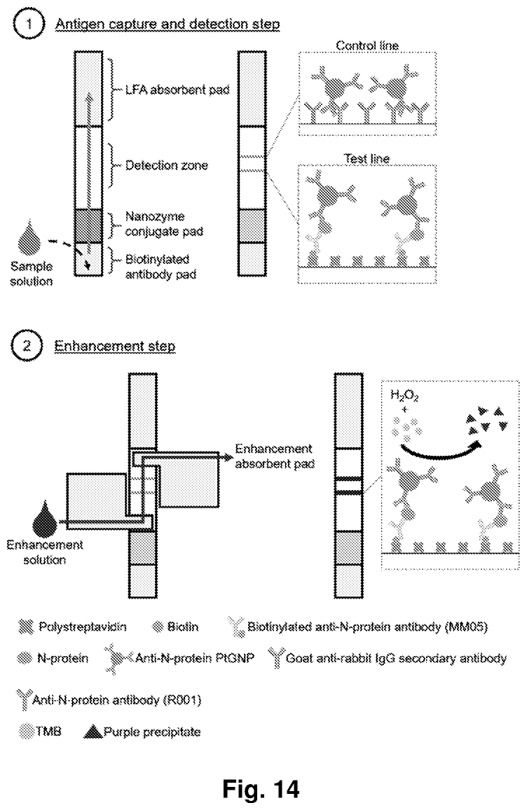

Operation of the device requires only two user steps: (1) adding the sample and (2) pushing down on the middle piece of the casing (e.g., by pushing down on the tabs or the push button) to move the middle piece from a first position to a second position. A simplified schematic of what occurs on the LFA test strip is shown in FIGS. 7 and 14. First, the patient sample is applied to the sample well (642 in FIG. 6, and 1342 in FIG. 13, panel A) in the casing (620 in FIG. 6 and 1320 in FIG. 13, panel A), which is located above the sample pad (102 in FIG. 1, 502 in FIG. 5, 1102a (biotinylated antibody pad) in FIG. 11). The liquid will therefore flow through the LFA test strip. This is the antigen capture detection step, where any analyte (e.g., N-protein) in the sample would become sandwiched between the antibody conjugates and the test line (110 in FIG. 1) which is disposed in a detection zone (113 in FIG. 1 and 1113 in FIG. 11).

Subsequently, the user would initiate the enhancement step by pressing down on the middle piece of the casing using the tabs 632a and 632b as shown in FIG. 6, or the push button 1350 as shown in FIG. 13, panel A. Lowering of the middle piece lowers the connector pads (430a and 430b in FIG. 4, and 1230a and 1230b in FIG. 12) (or where pads are absent, connector channels) to come into contact with the LFA test strip, creating a new flow path 746 as shown in shown in FIG. 7.

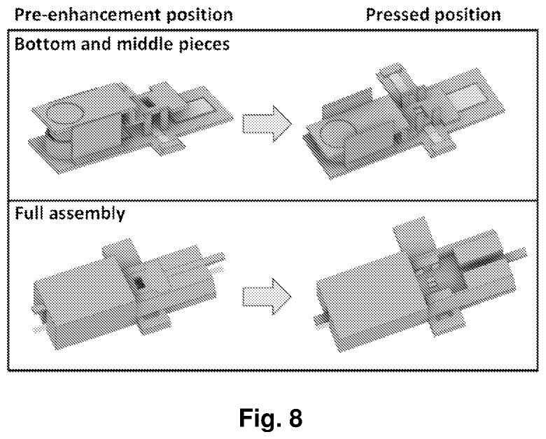

The buffer would also be released from the reservoir by puncturing of the reservoir seal (e.g., mylar foil) as the dome or cone (1154 in FIG. 11, panel A) is pressed into the seal (1229 in FIG. 12, panel A). This releases the enhancement reagent buffer from the buffer reservoir (1228 in FIG. 12, panel A) into the buffer well (1136 in FIG. 11, panel A). This buffer then rehydrates the enhancement reagents in the enhancement reagent pad(s) (538a and 538b in FIG. 5, and 1138a and 1138b in FIG. 11, panel A), flows through the lateral flow assay strip where it can react with the moieties at the test line and enhance a signal when the analyte is present, and flows into the second absorbent pad (enhancement reagent absorbent pad) 534 as shown in FIG. 5 and 1134 as shown in FIG. 11, panel A. As the enhancement reagents flow through the LFA strip, any of the PtGNPs bound to the test line catalyze the oxidation of the peroxidase substrate (e.g., TMB), ultimately resulting in the formation of a dark purple precipitate and an enhanced visible signal. The movement of the middle casing piece is further highlighted in FIG. 8 and in FIG. 13, panel B.

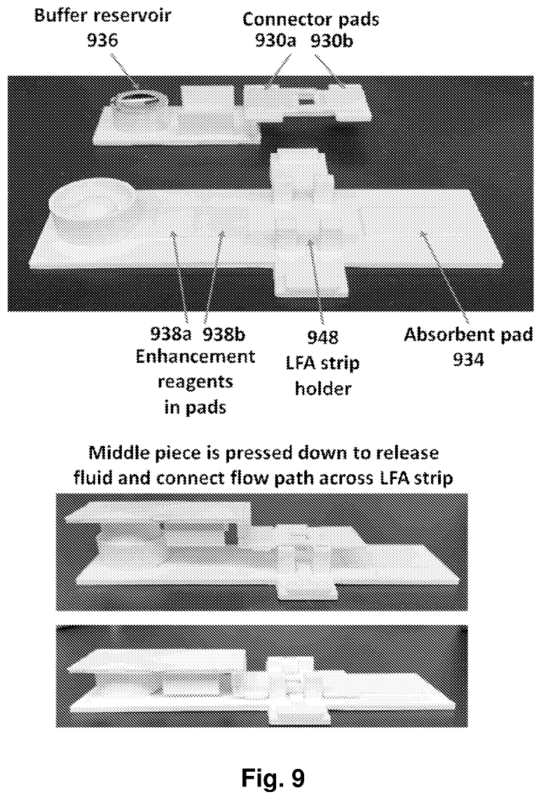

Prototypes of this casing were 3D printed out of a co-polyester plastic using an Ultimaker S3 3D printer. To hold fluid within the 3D printed reservoir, a buffer was pipetted into the reservoir and then the opening was heat sealed with a mylar foil. The buffer inside did not evaporate, leak out of the seal, or seep into the 3D-printed part over time, indicating the design is watertight and 3D printing of these pieces is a feasible option for low volume prototype manufacturing. An earlier design of the middle and bottom pieces of the casing can be seen in FIG. 9, along with the release of the liquid from the reservoir upon pressing down the middle piece.

In certain embodiments, to dehydrate the peroxide source required for the signal enhancement reaction on paper, sodium perborate tetrahydrate in Milli-Q water was applied to fully saturate glass fiber paper pads. These pads were then lyophilized for 2 hours. To dehydrate the TMB pads, 81.6 mM TMB DMF was diluted 20-60 fold in 0.1 M pH 5 citrate buffer. The solution was immediately applied to pieces of S17 fiberglass paper. For drying, these pads can be left in a vacuum-sealed desiccation chamber for 2 hours. Another method includes placing the pads on a hot plate or in an oven at 65° C. for 12 min until fully dry.

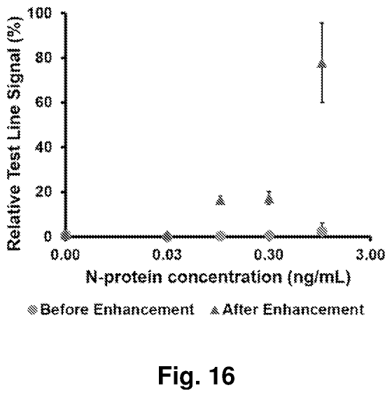

In certain embodiments, as illustrated herein in Example 1, urea hydrogen peroxide can be used as a peroxidase substrate. Different concentrations/numbers for the runs that led to FIGS. 15 and 16 are also described in Example 1.

While the disclosed example utilized platinum-coated gold nanozymes along with hydrogen peroxide and TMB to enhance the signal of the LFA, this invention is not limited to just this system. Nanozymes are well known to those of skill in the art, and the illustrated nanozymes could readily be replaced by nanozymes comprising materials such as platinum, gold, iron oxide, cerium-oxide, rubidium, iridium, copper, palladium, and combinations thereof. In certain embodiments the nanozyme comprises a nanoparticle selected from the group consisting of a platinum-coated gold nanoparticle, and Fe₃O₄ nanoparticle, a palladium core-shell nanoparticle, a Pt core shell nanoparticle, and a Pd/Pt core shell nanoparticle.

Similarly, the enhancement reagents can readily be replaced by other known enhancement reagents. Thus, for example, enzymes such as horseradish peroxidase and alkaline phosphatase, as well as gold and silver deposition reactions, could be utilized. Additionally, numerous other peroxidase substrates could be utilized. Such peroxidase substrates are well known to those of skill in the art and include, but are not limited to tetramethylbenzidine (TMB), diaminobenzidine (DAB), ABTS peroxidase substrate (Cas No: 28752-68-3), o-phenylenediamine dihydrochloride (OPD), and the like.

In certain embodiments substrates for alkaline phosphatase would include: nitroblue tetrazolium/5-bromo-4-chloro-3-indolyl phosphate (NBT/BCIP) and p-Nitrophenyl Phosphate, Disodium Salt (PNPP).

In certain embodiments for gold/silver enhancement, the reagent pad can contain gold (III) chloride or silver nitrate. Dehydrated in a separate pad would be a reducing agent such as L-ascorbic acid, hydroquinone, sodium citrate, or hydroxylamine. The detection antibodies would be conjugated to a gold nanoparticle to form the detection probe. The rehydrated enhancement reagents would react with the particle and deposit the gold or silver salts on the particle, where it will grow in size and enhance the visible signal.

The disclosed methods and devices improve the ease of use of these enhancement reactions on the LFA and subsequent integration of the casing and reactions with the LFA provide for the improved detection of the N-protein of SARS-CoV-2, or other analytes.

Moreover, in certain embodiments, different particles can be exploited to utilize their magnetic properties to allow immunomagnetic separation and preconcentration of the N-protein, which can also improve the detection limit. These particles will bind to the N-protein due to being decorated with antibodies for the N-protein and will subsequently be purified away from other molecules and concentrated with the use of magnets. One specific particle type is the iron oxide nanoparticles which are not only magnetic but also function as a nanozyme because they possess peroxidase-like behaviors and can catalyze signal enhancement reactions. In addition to magnetic metals that comprise or coat the particles polystyrene latex particles that encapsulate magnetic materials (e.g., iron oxide crystals) and colorimetric dyes can also be utilized. To ensure that these particles bound to the N-protein end up near the leading front of the moving fluid, magnets are appropriately placed in the device.

### EXAMPLES

The following examples are offered to illustrate, but not to limit the claimed invention.

### Example 1

### Nanozyme Signal Enhancement at the Push of a Button for the Improved Detection of SARS-CoV-2 Nucleocapsid Protein in Serum

In this example, we describe innovative LFA device comprising a casing that can be 3D printed casing and that incorporates a lateral-flow immunoassay, dehydrated signal enhancement reagents, and a sealed buffer chamber. With only the push of a button for signal enhancement, the device detected the SARS-CoV-2 N-protein in 40 min at concentrations as low as 0.1 ng/mL in undiluted serum.

### Materials and Methods

Preparation of Biotinylated Anti-N-Protein Capture Antibodies

All reagents and materials were purchased from Sigma-Aldrich (St. Louis, MO) unless otherwise noted. Biotinylated anti-N-protein capture antibodies were prepared by NHS-ester linkage using NHS-PEG-biotin. 15 μL of a 3 mM NHS-PEG-biotin solution was added to 50 μL of 0.5 mg/mL anti-N-protein antibodies (#40143-MM05, Sino Biological, Wayne, PA) in phosphate-buffered saline (PBS, pH 7.4) and reacted for 30 min, allowing the NHS-PEG-biotin to conjugate onto the free surface primary amines of the antibodies. The conjugation reaction was stopped via buffer exchange in fresh PBS using the Zeba Spin Desalting Columns.

Preparation of Anti-N-Protein Detection Antibody Decorated Platinum-Coated Gold Nanozyme Probes (Anti-N-Protein PtGNPs)

Platinum-coated gold nanozymes (PtGNs) were synthesized using a protocol derived from Gao et al. (2017) Nano Letts. 17:5572-5579. Briefly, 4 mL of 40 nm citrate-capped gold nanoparticles (GNs) (Nanocomposix, San Diego, CA) and 1686 μL of filtered ultrapure water were preheated to 90° C. in an oil bath under magnetic stirring for 20 min. Following the preheating, 314 μL of a 0.82 mM chloroplatinic acid hydrate solution and 2 mL of a 3.3 mM ascorbic acid solution were injected separately into the gold nanoparticle suspension using a syringe pump at rates of 0.6 and 1.2 mL/h, respectively. The reaction was allowed to proceed for 1 h after the injection was complete.

To create anti-N-protein decorated platinum-coated gold nanozyme probes (anti-N-protein PtGNPs), 30 μL of a 0.1 M sodium borate solution (pH 9) was first added to 1 mL of PtGNs. Then, 4 μg of primary anti-N-protein antibody (#40143-R001, Sino Biological, Wayne, PA) was added to the suspension and incubated for 30 min at room temperature (22° C.). 50 μL of a 10% (w/v) bovine serum albumin (BSA) in filtered ultrapure water solution was then added to the suspension and incubated for 10 min. Free antibodies were removed with three centrifugation cycles at 8600 RCF and 4° C. for 6 min each. For the first two cycles, the pellet was resuspended in 200 μL of 1% (w/v) BSA in filtered ultrapure water, and the final pellet was resuspended in 50 μL of a 0.1 M sodium citrate buffer (pH 6) solution.

Preparation of LFA Test Strip

The LFA test strips were composed of overlapping pads secured to an adhesive backing. These pads included a biotinylated-anti-N-protein antibody pad, an anti-N-protein PtGNP conjugate pad, a nitrocellulose membrane, and a CF4 absorbent pad (Cytiva, Marlborough, MA). To prepare the detection region of the LFA, proteins were first printed and immobilized on a Unisart CN140 nitrocellulose membrane (Sartorius, Göttingen, Germany) using an Automated Lateral Flow Reagent Dispenser (Claremont BioSolutions LLC, Upland, CA) with the voltage setting at 4.5 V and a Fusion 200 syringe pump (Chemyx Inc, Stafford, TX) with a flow rate of 300 μL/min. The test line was formed by printing a solution of a 2 mg/mL polystreptavidin (Biotez, Berlin, Germany) solution in 25% (w/v) sucrose. The control line was formed by printing a solution of 0.25 mg/mL goat anti-rabbit IgG secondary antibody in 25% (w/v) sucrose. The printed membrane was left in a vacuum-sealed desiccator overnight and subsequently stored in a bag containing Drierite desiccant (W.A Hammond Drierite Co, Xenia, OH) for an additional day.

To create each nanozyme conjugate pad, 6 μL of anti-N-protein PtGNPs were diluted to form a 20 μL solution with final concentrations of 5% (w/v) trehalose and 1% (w/v) BSA and then dehydrated onto a 5 mm×10 mm piece of Standard 17 fiberglass paper (Cytiva). The conjugate pads were dehydrated in a desiccator at 37° C. overnight. To create each capture antibody pad, 2 μL of a 0.05 mg/mL biotinylated anti-N-protein capture antibody solution was diluted to form a 20 μL solution with final concentrations of 5.74% (w/v) trehalose and 1.15% (w/v) BSA and then dehydrated onto a 5 mm×10 mm piece of fiberglass paper. The pads were dehydrated in a vacuum-sealed desiccator overnight.

To assemble the LFA test strip, the nitrocellulose membrane was first adhered to an adhesive backing. Individual strips were cut to be 5 mm in width. To each strip, a CF4 absorbent pad was placed on the adhesive backing downstream of the control line, overlapping the nitrocellulose membrane by 3 mm. The PtGNP conjugate pad was placed on the adhesive backing upstream of the test line, overlapping the nitrocellulose membrane by 2 mm. The biotinylated capture antibody pad was placed on the adhesive backing upstream of and overlapping the PtGNP conjugate pad by 1 mm.

Design and Assembly of Device for Enhancement Reagent Storage and Delivery on LFA

A casing was designed to eliminate the need for multiple liquid- and test strip-handling steps. This 3D printed device provides in-test liquid reagent storage, dehydrated enhancement reagents, and movable paper architecture that directs the flow of liquid through the LFA test strips. The three major components of the device are outlined in FIG. 10. The parts shown in gray were 3D printed using an Ultimaker 3 FDM 3D printer (Ultimaker B.V., Geldermalsen, Netherlands) out of Ultimaker CPE filament (co-polyester).

The bottom piece of the casing along with the inserted paper pads and test strip are detailed in FIG. 11, panels A and B. In the illustrated embodiment, the enhancement buffer release well is an enclosed, hollow cylinder with a dome in the center. The dome serves to rupture a foil sealed buffer reservoir on the middle piece of the casing. 0.05 g of urea hydrogen peroxide was sprinkled in the hollow cylinder surrounding the dome and then covered with a ring of fiberglass paper. A 62.5 μL solution of 6.5 mM 3,3′,5,5′-tetramethylbenzidine (TMB), 15% (w/v) trehalose, and 20% (w/v) dimethylformamide in 0.1 M sodium citrate buffer (pH 5) was dehydrated onto a 13 mm×12 mm fiber glass pad overnight in a vacuum sealed desiccator to create the TMB pad. The enhancement reagent absorbent pad is composed of a 13 mm×23 mm CF4 absorbent pad. The four aligning snap fit joints hold the middle piece of the casing in a lifted position until the user presses down on it. When pressed, the middle piece then snaps into place and is held down in a constant position by the snap fit joints. The bottom piece also contains a sample well which is located above the biotinylated capture antibody pad when the device is fully assembled.

The movable middle piece of the casing, shown in FIG. 12, panels A and B, contains the enhancement buffer reservoir and two connector pads. The left pad is made up of Standard 17 fiberglass paper while the right pad is a CF4 absorbent pad. The enhancement buffer, which will solubilize the urea hydrogen peroxide and TMB during the assay, was stored within the reservoir of the middle piece. To fill the reservoir, 600 μL of 1% (w/v) dextran sulfate in 0.1 M sodium citrate buffer (pH 5) was pipetted into the reservoir. To seal the liquid in the reservoir, a sheet of mylar foil was placed on top of the reservoir and heat was applied using a hot iron for 3 s followed by complete cooling. The top piece of the casing serves to help hold the other components in place and protect them from external and environmental factors. It also contains a viewing window to observe the detection results (FIG. 13, panel A).

Detection of N-Protein in Human Serum with Nanozyme Signal Enhanced LFA

To detect for N-protein using our nanozyme signal enhanced LFA, a 25 μL human serum sample containing varying concentrations of N-protein was added to the sample well on the LFA (above the biotinylated capture antibody pad). As in the case of typical LFAs for serum samples, this was immediately followed by a chase buffer. In our system, we used 75 μL of chase buffer composed of 2% (w/v) polyvinylpyrrolidone 10 kDa, 0.2% (w/v) BSA, 0.2% (w/v) Tween 20, and 0.2% (w/v) casein in 0.1 M potassium phosphate at pH 7.2. After 20 min, the user pressed the button to move down the middle piece of the casing (FIG. 13, panel B). The movement of the middle casing piece resulted in the rupture of the mylar seal to release the enhancement buffer and also served to lower the connector pads to provide a continuous flow path for the enhancement reagents to flow through the LFA strip. Final results were observed after 20 min of enhancement. Results were photographed before and after the signal enhancement reaction with a Nikon D3400 digital camera (Nikon, Tokyo, Japan) in a controlled lighting environment. To quantify the relative test line intensities, the resulting images were processed by a MATLAB script developed by our lab (Jue et al. (2014) Biotechnol. & Bioengin. 111: 2499-2507).

### Results and Discussion

Demonstration of Improved N-Protein Detection Using Nanozyme Signal Enhancement

The operation of our device for the nanozyme signal enhanced detection of N-protein occurs in two main steps. The first is the antigen capture and detection step and the second is the signal enhancement step (FIG. 14). The user first applies the serum sample to the sample well immediately followed by the addition of the chase buffer. The liquid will first resolubilize the biotinylated capture anti-N-protein antibody and then the anti-N-protein PtGNPs. In the case of a positive sample, these antibody species will bind to any N-protein in the sample resulting in the formation of sandwich complexes. As these complexes flow through the LFA strip, they will be captured at the test line due to the strong biotin-streptavidin interaction between the biotinylated capture antibody and the streptavidin immobilized on the test line. This will ultimately result in the capture of PtGNPs at the test line region. In the case of a negative sample where no N-protein is present, no sandwich complex will form. Therefore, even though the biotinylated capture antibody will bind to the streptavidin at the test line, no PtGNPs will be captured. Regardless of the sample being positive or negative for N-protein, any PtGNPs that do not get captured at the test line will be able to be captured by the secondary antibody at the control line to indicate that the sample flowed properly through the test strip.

After 20 min, instead of the typical signal enhancement process of a user creating a signal enhancement solution and then physically moving the LFA strip into that solution, the user will only need to press down on the button connected to the middle piece of the casing. This lowers the middle piece where it snaps into place with the connector pads bridging gaps between the dehydrated TMB pad and the LFA strip, as well as the LFA strip and the enhancement absorbent pad. Additionally, as the middle piece is lowered, the mylar seal on the enhancement buffer reservoir becomes ruptured by the dome, which allows the enhancement buffer to flow into the release well. Once released, the buffer solubilizes the urea hydrogen peroxide, followed by the TMB. This enhancement solution then flows through the LFA test strip and into the enhancement absorbent pad. As the solution passes the detection zone, any PtGNPs bound to the test line will catalyze the oxidation of TMB to TMB⁺. The TMB⁺ will complex with the negatively charged dextran sulfate, leading to the formation of an insoluble purple product that becomes deposited at the test line. This results in the enhancement of the test line signal over an additional 20 min, improving the sensitivity of the LFA.

To evaluate the performance of this assay, we tested samples containing 0, 0.03, 0.1, 0.3 and 1 ng/mL of N-protein spiked into human serum. The final LFA strips from one of our experimental studies are shown in FIG. 15. Before the enhancement step, a clearly visible test line is present at 1 ng/mL but not at 0.3 ng/mL, indicating a detection limit of 1 ng/mL. After enhancement, the test line at 1 ng/mL becomes significantly darker and a visible test line also appears at 0.1 and 0.3 ng/mL, demonstrating at least a 10-fold improvement in detection limit.

This experimental study was performed three times. The relative test-line intensities were then quantified using a custom MATLAB script developed by our lab, and the results are shown in FIG. 16. These results demonstrate the ability of our nanozyme signal enhanced assay to consistently detect for N-protein in serum down to 0.1 ng/mL. This is at least a 10-fold improvement over the conventional LFA as the enhanced signal at 0.1 ng/mL is significantly greater than the unenhanced signal at 1 ng/mL. This current detection limit falls within the physiologically relevant range of serum N-protein concentrations reported for SARS-CoV-2 (Che et al. (2004) Emerging Infect. Dis. 10: 1947-1949; Wang et al. (2020) Front. Cell. Infection Microbiol. 10: 470; Shan et al. (2021) Nat. Comm. 12: 1931). Additionally, the detection limit of 0.1 ng/mL matches that of a recently developed smartphone-based microfluidic device despite our device not having any electronic components (Li and Lillehoj (2021) ACS Sensors, 6: 1270-1278).¹² Besides serum N-protein detection, our signal enhanced assay would be suitable for the detection of N-protein in swab-based samples and could also be adapted for the detection of SARS-CoV-2 spike protein or other antigen targets.

While our device has a more complex construction than the conventional LFA, the casing can still be mass produced using injection molding processes and the test strips can be created using existing LFA diagnostic manufacturing infrastructure. Moreover, compared to other approaches to improve sensitivity such as the integration of electronic readers, our device is much less complex, making it easier to scale-up production and be more affordable to the end user (Peng et al. (2020) Appl. Phys. Lett. 117: 2-5). The steps for operation are also not much more difficult than the conventional LFA, requiring just an additional press of a button.

### CONCLUSIONS

In summary, we have developed a nanozyme signal enhanced LFA for the improved detection of the N-protein of SARS-CoV-2 in serum. An innovative 3D printable casing was designed, which stored all assay components including the LFA test strip, dehydrated signal enhancement reagents, and a sealed chamber with stored liquid enhancement buffer. Our paper-based device was able to detect N-protein in undiluted serum in 40 min at concentrations as low as 0.1 ng/mL, which was at least a 10-fold improvement over the conventional LFA. Moreover, with this all-in-one device, only one simple step of pushing a single button is needed for the signal enhancement to occur after the LFA detection step. The development of devices that have the ability to detect for SARS-CoV-2 antigen biomarkers with improved sensitivity, while maintaining a user-friendly design and scalable manufacturing, is vital to increasing the frequency in screening asymptomatic individuals. This has the potential to significantly improve the response to the COVID-19 pandemic by effectively detecting patients at their early stages of infection and allowing for effective treatment and quarantining procedures to be implemented.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A device for performing a signal-enhanced lateral flow immunoassay to detect an analyte, said device comprising:
a case comprising a top piece, a middle piece, and a bottom piece where said casing is configured so provide that the middle piece can move from a first position to a second position, wherein:
said bottom piece comprises a lateral flow immunoassay strip configured to detect said analyte, one or more enhancement reagent pads comprising enhancement reagent(s) disposed on or within said enhancement reagent pads, an enhancement reagent absorbent pad configured to receive said enhancement reagent(s), and a well or receptacle configured to deliver a buffer into said one or more pads comprising enhancement reagent(s);
said top piece comprises an opening to a sample well for receiving a sample and a viewing window that permits viewing of a detection zone on said lateral flow immunoassay strip; and
said middle piece comprises a reservoir that contains an enhancement reagent buffer, one or more connector channels and/or connector pads configured to carry said enhancement reagent buffer and said one or more enhancement reagents to and across said lateral flow immunoassay strip to said enhancement reagent absorbent pad, and one or more tabs or a button to move said middle piece from said first position to said second position; and wherein:
in said first position, said channels and/or connector pads are not in fluid communication with said lateral flow immunoassay strip and said reservoir is sealed; and
in said second position, said reservoir is in fluid communication with well which is in fluid communication with said one or more channels and/or connector pads, said one or more pads comprising enhancement reagents, and said lateral flow immunoassay strip and thereby capable of delivering said enhancement reagent buffer and said one or more enhancement reagents to said lateral flow immunoassay strip.

2. The device of claim 1, wherein said analyte comprises a nucleocapsid protein (N-protein) of a corona virus.

3. The device of claim 2, wherein said analyte comprises a nucleocapsid protein (N-protein) of SARS-CoV-2 (Covid 19).

4. The device according to any one of claims 1-3, wherein said lateral flow assay strip comprise a sample pad, a detection zone downstream from the sample pad, and an absorbent pad downstream from the detection zone, and where said detection zone comprises a test line comprising biotin or avidin.

5. The device of claim 4, wherein said lateral flow assay strip comprises a capture moiety disposed in said LFA strip upstream from said detection zone and said capture moiety comprises a first anti-analyte antibody conjugated to a biotin when said test line comprises an avidin and said capture moiety comprises a first anti-analyte antibody conjugated to an avidin when said test line comprises a biotin.

6. The device of claim 5, wherein said anti-analyte antibody is conjugated to a biotin and said test line comprises an avidin.

7. The device of claim 6, wherein said test line comprises streptavidin, or polystreptavidin.

8. The device according to any one of claims 4-7, wherein said detection zone comprises a control line functionalized to capture an antibody.

9. The device of claim 8, wherein said control line comprises an anti-IgG antibody.

10. The device according to any one of claims 5-9, wherein said capture moiety is dried onto or into said lateral flow assay strip or onto or into a pad disposed on said lateral flow assay strip.

11. The device according to any one of claims 5-10, wherein said capture moiety is disposed on said lateral flow assay strip between said sample pad and said detection zone or on a pad (e.g., nanozyme conjugate pad) disposed on said lateral flow assay strip between said sample pad and said detection region.

12. The device according to any one of claims 5-10, wherein said capture moiety is disposed on or in said sample pad.

13. The device according to any one of claims 5-12, wherein said first anti-analyte antibody comprises an anti-N-protein antibody.

14. The device according to any one of claims 5-12, wherein said lateral flow assay comprises a region containing an indicator conjugate comprising an indicator moiety attached to a second anti-analyte antibody or wherein said lateral flow assay comprises a conjugate pad (e.g., nanozyme conjugate pad) containing said indicator conjugate.

15. The device of claim 14, wherein said second anti-analyte antibody comprises an anti-N-protein antibody.

16. The device according to any one of claims 14-15, wherein said indicator moiety comprises an indicator selected from the group consisting of a nanozyme, horseradish peroxidase, alkaline phosphatase, and a gold nanoparticle.

17. The device according to any one of claims 14-16, wherein said indicator conjugate is disposed on said lateral flow assay strip between said sample pad and said detection region or on a pad (e.g., nanozyme conjugate pad) disposed on said lateral flow assay strip between said sample pad and said detection region.

18. The device according to any one of claims 14-17, wherein said indicator conjugate is disposed on or in said lateral flow assay strip downstream from said capture moiety or on or in a pad disposed on said lateral flow downstream from said capture moiety.

19. The device according to any one of claims 1-18, wherein said bottom piece comprises one pad with one or more enhancement reagent(s) disposed on or within said pad.

20. The device according to any one of claims 1-18, wherein said bottom piece comprises two pads with one or more enhancement reagent(s) disposed on or within each pad.

21. The device according to any one of claims 19-20, wherein the enhancement reagent(s) are dried onto said enhancement reagent pad(s).

22. The device according to any one of claims 16-21, wherein said indicator conjugate comprises a nanozyme conjugate comprising a nanozyme attached to said second anti-analyte antibody.

23. The device of claim 22, wherein said nanozyme comprises a material selected from the group consisting of platinum, gold, iron oxide, cerium-oxide, rubidium, iridium, copper, and palladium.

24. The device of claim 23, wherein said nanozyme comprises a nanoparticle selected from the group consisting of a platinum-coated gold nanoparticle, and Fe₃O₄ nanoparticle, a palladium core-shell nanoparticle, a Pt core shell nanoparticle, and a Pd/Pt core shell nanoparticle.

25. The device of claim 24, wherein said nanozyme comprises a platinum-coated gold nanoparticle.

26. The device according to any one of claims 22-25, wherein said one or more enhancement reagent pads contains a peroxidase substrate selected from the group consisting of tetramethylbenzidine (TMB), diaminobenzidine (DAB), ABTS peroxidase substrate (Cas No: 28752-68-3), and o-phenylenediamine dihydrochloride (OPD), chemiluminescent luminol, a xanthan ester, and an acridan-based reagent.

27. The device of claim 26, wherein said peroxidase substrate comprises TMB.

28. The device according to any one of claims 16-21, wherein said indicator moiety comprises alkaline phosphatase.

29. The device of claim 28, wherein said one or more enhancement reagent pads contains a substrate selected from the group consisting of nitroblue tetrazolium/5-bromo-4-chloro-3-indolyl phosphate (NBT/BCIP), and p-Nitrophenyl Phosphate, Disodium Salt (PNPP).

30. The device according to any one of claims 16-21, wherein said indicator moiety comprises a gold nanoparticle.

31. The device of claim 30, wherein said one or more enhancement reagent pads contains a substrate selected from the group consisting of gold (III) chloride, and silver nitrate.

32. The device according to any one of claims 1-31, wherein said lateral flow assay strip, said LFA absorbent pad, said LFA sample pad, said one or more reagent pads, and said enhancement reagent absorbent pad each comprise a paper.

33. The device of claim 32, wherein said the paper comprising wherein said lateral flow assay strip, said LFA absorbent pad, said LFA sample pad, said one or more reagent pads, and said enhancement reagent absorbent pad are independently selected from the group consisting of a cellulose paper, a fiberglass paper, a nitrocellulose membrane, a polyvinylidene fluoride, a nylon membrane, a charge modified nylon membrane, a polyethersulfone, and a cotton linter material (e.g., CF4), and combinations thereof.

34. The device according to any one of claims 32-33, wherein said lateral flow assay strip comprises a nitrocellulose membrane.

35. The device according to any one of claims 32-34, wherein lateral flow assay absorbent pad comprises a cotton linter material (e.g., CF4).

36. The device according to any one of claims 32-35, wherein said sample pad comprises a fiberglass paper.

37. The device according to any one of claims 32-36, wherein said indicator conjugate is disposed in a pad comprising a fiber glass paper.

38. The device according to any one of claims 32-37, wherein said enhancement reagent pad(s) each comprise a fiberglass paper.

39. The device according to any one of claims 1-38, wherein:
said middle piece comprises a first connector pad configured so that, when said middle piece is in the second position said first connector pad is upstream from said lateral flow assay strip with respect to the flow of said enhancement reagent buffer and provides fluid communication from said enhancement reagent pad(s) to said lateral flow assay strip; and
said middle piece comprises a second connector pad configured so that, when said middle piece is in the second position said second connector pad is downstream from said lateral flow assay strip with respect to the flow of said enhancement reagent buffer and provides fluid communication from said lateral flow assay strip to said enhancement reagent absorbent pad.

40. The device of claim 39, wherein said first connector pad and said second connector pad each comprise a paper.

41. The device of claim 40, wherein said the paper comprising said first connector pad and said second connector pad are independently selected from the group consisting of a cellulose paper, a fiberglass paper, a nitrocellulose membrane, a polyvinylidene fluoride, a nylon membrane, a charge modified nylon membrane, a polyethersulfone, and a cotton linter material (e.g., CF4), and combinations thereof.

42. The device according to any one of claims 40-41, wherein said first connector pad comprises a fiberglass paper.

43. The device according to any one of claims 40-42, wherein said second connector pad comprises a cotton linter material.

44. The device according to any one of claims 1-38, wherein:
said middle piece comprises a first connector channel configured so that, when said middle piece is in the second position said first connector channel is upstream from said lateral flow assay strip with respect to the flow of said enhancement reagent buffer and provides fluid communication from said enhancement reagent pad(s) to said lateral flow assay strip; and
said middle piece comprises a second connector channel configured so that, when said middle piece is in the second position said second connector channel is downstream from said lateral flow assay strip with respect to the flow of said enhancement reagent buffer and provides fluid communication from said lateral flow assay strip to said enhancement reagent absorbent pad.

45. The device according to any one of claims 1-44, wherein said well or receptacle configured to deliver a buffer into said one or more pads comprising enhancement reagent(s) comprises is an enclosed hollow cylinder with a dome or cone in the center configured to puncture a seal on said reservoir when said middle piece is in the second position and thereby release enhancement reagent buffer into said well or receptacle.

46. The device according to any one of claims 1-45, wherein said reservoir is sealed with a foil seal.

47. The device according to any one of claims 1-46, wherein said top piece and said bottom piece are snapped together.

48. The device according to any one of claims 1-47, wherein said middle piece comprises a push button that protrudes through said top piece and operates to move said middle piece from said first position to said second position.

49. The device according to any one of claims 1-47, wherein said middle piece comprises tabs that protrude through sides of said device said and operates to move said middle piece from said first position to said second position.

50. A method of detecting an analyte, said method comprising:
applying a sample comprising said analyte to a lateral flow assay configured for the capture of said analyte at a test line and permitting said analyte to flow through said lateral flow assay; and
contacting said lateral flow with signal enhancement reagents to enhance and improve visibility of a signal at said test line.

51. The method of claim 50, wherein said enhancement reagents comprise a nanozyme.

52. The method according to any one of claims 50-51, wherein said method is performed using a device according to any one of claims 1, and 4-49, wherein said method comprises:
applying said sample to a sample well on said device when said second piece is in said first position;
allowing said sample to pass through said lateral flow assay; and
moving said second piece from said first position to said second position and allowing said enhancement reagents to react on bound enhancement reagents (e.g., nanozyme) on said test line; and
detecting a visible line at said test line that indicates the presence of said analyte.

53. The method of claim 52, wherein said analyte comprises a nucleocapsid protein (N-protein) of a corona virus.

54. The method of claim 53, wherein said analyte comprises a nucleocapsid protein (N-protein) of SARS-CoV-2 (Covid 19).

55. The method according to any one of claims 50-54, wherein said sample comprises a biological sample.

56. The method of claim 55, wherein said sample comprises a biological sample selected from the group consisting of a culture, blood, serum, saliva, nasal mucus, cerebral spinal fluid, urine, stool, bronchial aspirates, tracheal lavage, pleural fluid, milk, lymph, sputum, semen, needle aspirate, punch biopsy, and surgical biopsy.

57. The method of claim 56, wherein said sample comprises a biological sample selected from the group consisting of nasal mucus, oral fluid, bronchial aspirate, and trachial lavage.
